(19) United States
(12) Patent Application Publication
(10) Pub. No.: US 2012/0238807 A1
(43) Pub. Date: Sep. 20, 2012

(54) ENDOSCOPY SYSTEM

(76) Inventors: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP)

(21) Appl. No.: 13/361,259

(22) Filed: Jan. 30, 2012

(30) Foreign Application Priority Data
Mar. 15, 2011 (JP) 2011-056344

(51) Int. Cl. A61B 1/04

(52) U.S. Cl. 600/109

(57) ABSTRACT

An endoscopy system includes a self-propelling endoscope attached with a self-propelling device, a drive source device for providing drive force to the self-propelling device, a controller connected to the drive source device and for controlling moving direction and speed of the self-propelling device, and a monitor for displaying a captured image. The drive source device transmits drive information such as forward movement, backward movement, stop, speed, load torque of the self-propelling device to a processing device. The processing device displays the drive information on the monitor with the captured image.

### FIELD OF THE INVENTION

The present invention relates to an endoscopy system having a self-propelling endoscope, and more particularly to an endoscopy system which enables to confirm the self-propelling state.

### BACKGROUND OF THE INVENTION

An endoscope is widely used for medical examination. The endoscope has an insert section and an operating section. In the medical examination, the insert section of the endoscope is inserted into a body cavity of patient, and an image of the body cavity captured through an imaging window provided at a front end portion of the insert section is displayed on a monitor. The direction and position of the front end portion are changed during observing the monitor, and detection, diagnosis, treatment of pathologic lesion is performed.

In colorectal examination, manipulation of the insert section is generally difficult. This is because a large colon twists and curves, additionally diameter thereof is not uniform, so that much experience of manipulation of the endoscope is required. Particularly, since S-moid colon and transverse colon are not fixed in the body cavity, their shapes and positions changes within the length thereof, and they are deformed by contact force when the endoscope is inserted. Then, the insert section is gradually inserted into the intestine canal during repeating forward movement and backward movement of the endoscope, so that the long time is required for inspection and the physical load to the body of a patient is very large.

In recent years, a self-propelling endoscope which enables easy insertion of the insert section into the intestine canal even for an operator who is insufficiently skilled in the manipulation is suggested. WO 2007/050370 discloses a self-propelling device attached to the front end portion of the endoscope, for assisting insertion of the insert section in the body cavity. The self-propelling device disclosed in the WO 2007/050370 generates small friction force between the intestine wall and the device, and pulls the intestine colon to be in an almost straight line when the intestine colon located in a moving direction curls and bends, so that a pathway for the endoscope is obtained. Therefore, in the part of the intestine wall fixed to the body cavity, the self-propelling device is neither allowed to pull the intestine colon nor to move, so that the operator needs to operate the endoscope. Since the self-propelling device does not work at some part in the body cavity, the operator must see the display of the driving device each time and check the operating condition of the driving device as to whether the driving device works as indicated or not. When checking the operating condition of the driving device, the operator must remove his/her gaze from the monitor, which cause troublesome in the operation.

Moreover, a foot operated switch is commonly used as a directing device for directing drive/stop, insertion/removal of the self-propelling device. Since the operator gazes the monitor displaying the endoscope image on a constant basis, there is a concern that the operator incorrectly foots the foot operated switch. Also in this case, the operator can not find the incorrect operation unless the operator checks the display of the driving device.

### SUMMARY OF THE INVENTION

An object of the present invention is to provide an endoscopy system which enables to check operating state of a drive source device without removing his/her gaze from a monitor screen.

In order to achieve the above object and others, the endoscopy system of the present invention is provided with a self-propelling endoscope, a drive source device, a controller, a monitor. The self-propelling endoscope includes an insert section which is self-propulsive in the body cavity, and inserted into the body cavity, a self-propelling device provided at a front end portion of the insert section, and an image capturing section contained in the front end portion. The self propelling device provides self propulsion to the insert section to perform forward and backward movement, and the image capturing section captures image of inside of the body cavity. The drive source device supplies drive force for self-propulsion to the self-propelling device.

The controller controls operation of the self-propelling device through the drive source device. The monitor displays the observed image captured at the image capturing section and the drive information of the drive source device.

Moreover, it is preferable that the drive information is displayed on the monitor with the observed image, and a recording device for recording a screen displayed on the monitor is further included. The drive information preferably includes the forward movement and the backward movement, and they are displayed by at least one of arrow or character, and arrows are displayed at four corners of an image display area on which the observed image is displayed. Preferably, four arrows displayed at four corners of the image display area direct toward a center part of the image display area when drive force toward the front end portion is in the direction of forward movement, and the four arrows direct toward outside of the image display area when drive force toward the front end portion is in the direction of backward movement. The drive information preferably includes at least one of moving velocity and load torque of the front end portion corresponding to the drive force, an alarm for informing the forward movement and said backward movement by different sounds is preferably provided.

According to the present invention, drive information in relation to the drive source device providing drive force to the self-propelling device is displayed on the monitor screen with the observed image, so that operating state of the drive source device, i.e. operating state of the self-propelling device can be checked without remove his/her eyes from the monitor screen.

### BRIEF DESCRIPTION OF THE DRAWINGS

The above objects and advantages of the present invention will become more apparent from the following detailed description when read in connection with the accompanying drawings, in which:

FIG. 1 is an external view showing an endoscopy system;

FIG. 2 is a side view showing a self-propelling endoscope;

FIG. 3 is an enlarged view of a front end part of the self-propelling endoscope;

FIG. 4 is a block diagram of the endoscopy system;

FIG. 5 is a view showing display screen of a monitor displaying forward movement state;

FIG. 6 is a view showing display screen of a monitor displaying backward movement state;

FIG. 7 is a view showing a forward movement state by character in a similar manner to FIG. 5;

FIG. 8 is a view showing a backward movement state by character in a similar manner to FIG. 6; and

FIG. 9 is a view showing other drive information in a similar manner to FIG. 7.

### DESCRIPTION OF THE PREFERRED EMBODIMENTS

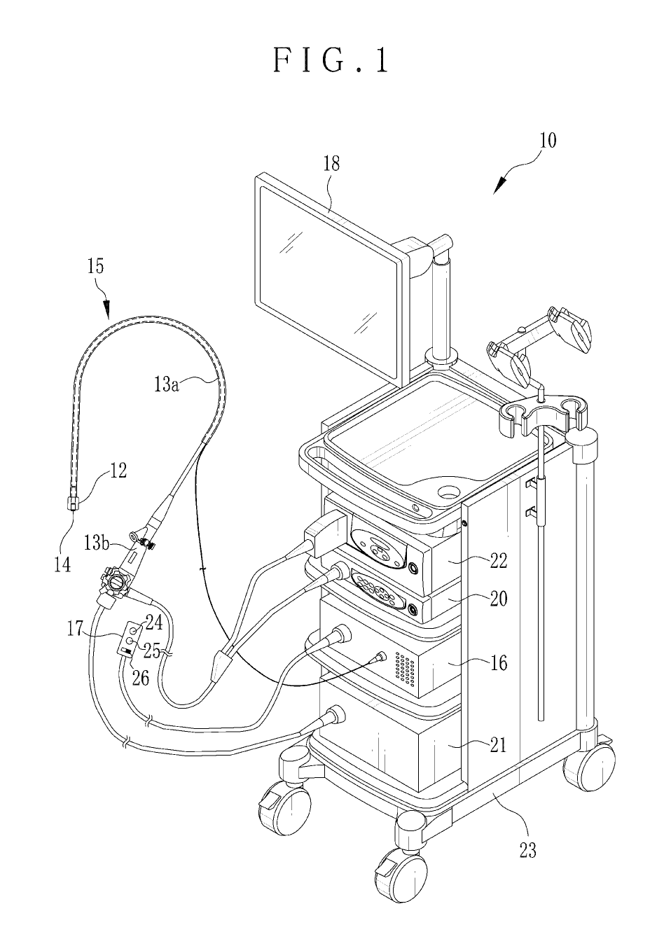

In FIG. 1, an endoscopy system 10 of the present invention includes a self-propelling endoscope 15 (hereinafter referred to as an endoscope), a drive source device 16 for supplying drive force for self-propulsion to the endoscope 15, a processing device 20 for controlling the endoscope 15, a light source device 22 for supplying illumination light to the endoscope 15, a transfusion device 21, a controller 17 for controlling self-propulsion of the endoscope 15, a monitor 18 for displaying an operation screen and an observed image and so on. They are loaded on an endoscope cart 23 with wheels.

The endoscope 15 is provided with an insert section 13a inserted into a body cavity, for example, digestive tract such as large intestine, and provided with an operating section 13b for operating the insert section 13a. A self-propelling device 12 for assisting movement of a front end portion 14 in the digestive tract is detachably attached to the front end portion 14 of the insert section 13a. A self-propelling device described in WO 2007/050370 is used as the self-propelling device 12.

The controller 17 includes a forward button 24 for instructing forward movement of the self-propelling device 12, a backward button for instructing backward movement thereof, a speed setting button 26 for setting speed. Further, the transfusion device 21 supplies cleaning water to the endoscope 15 to wash the front end portion 14.

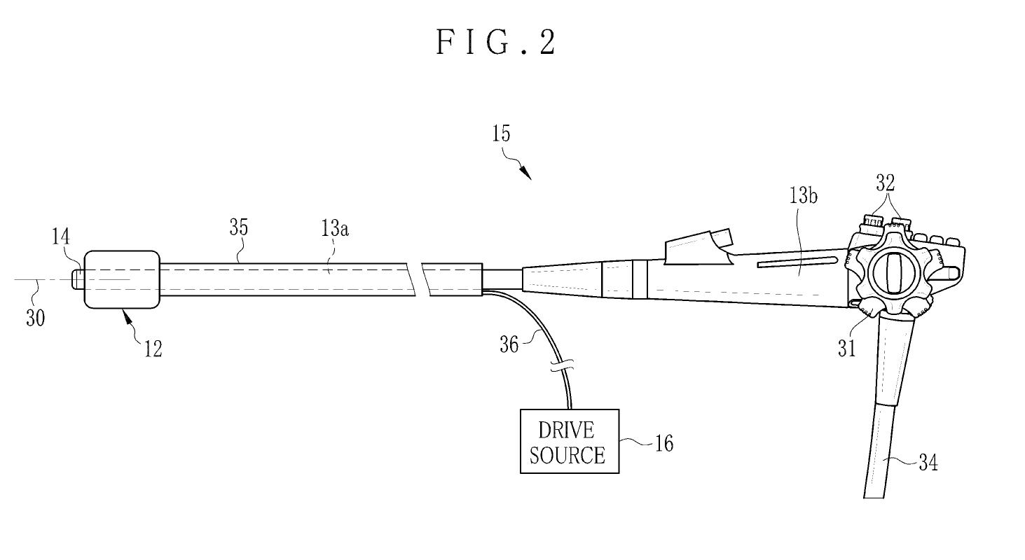

In FIG. 2, the operating section 13b of the endoscope 15 is provided with an angle knob 31 for changing the direction of the front end portion 14, and an operating button 32 for supplying air/water. Further, the operating section 13b has a universal code 34 connecting to the processing device 20 and the light source device 22. The universal code 34 is incorporated with an air/water channel, a cable for outputting image signal, and a light guide.

An overtube 35 is fit onto the insert section 13a and a protective sheath 36 is inserted between the insert section 13a and the overtube 35. The overtube 35 is formed in an accordion-fold shape elastically expandable in a direction of the axis 30 of the insert section 13a.

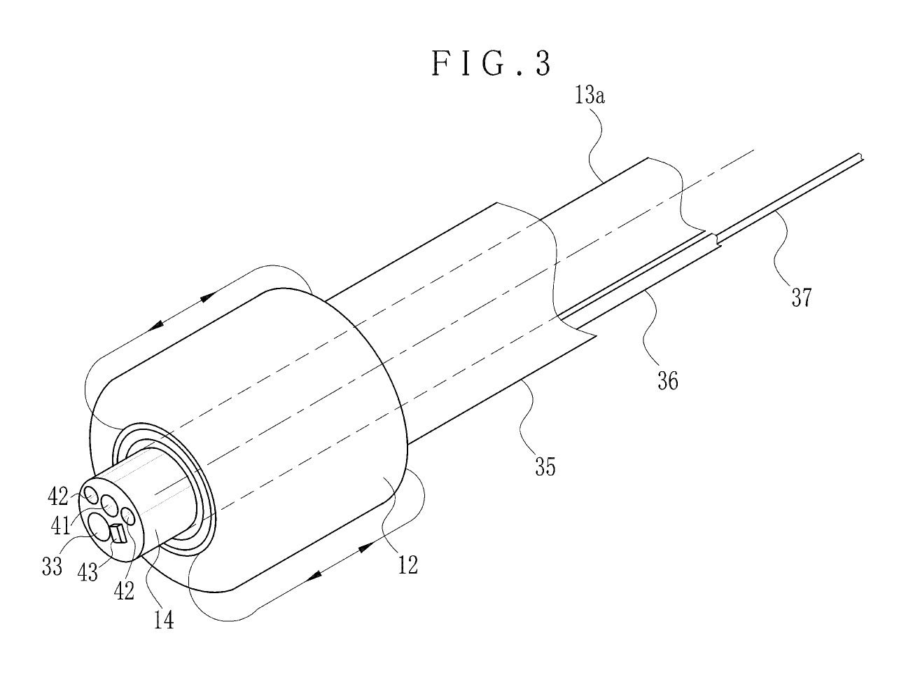

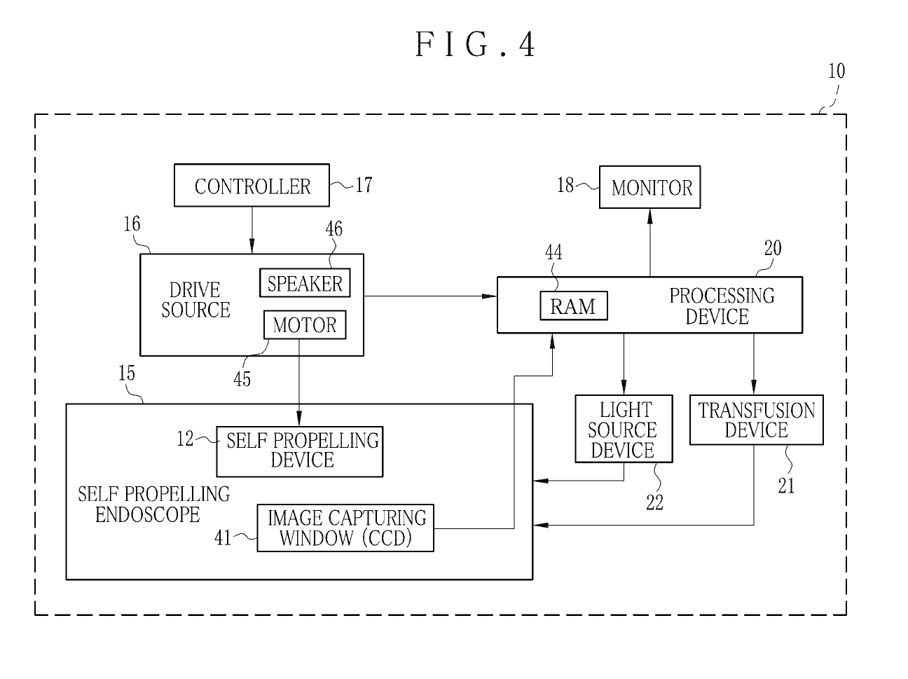

In FIG. 3, the insert section 13a is in rod-like form having flexibility, a front surface of the front end portion 14 is provided with an image capturing window 41, an illumination window 42, an air/water nozzle 43, a forceps outlet 33 and so on. Imaging optical system solid-state imaging elements (CCD sensor, CMOS sensor etc.) are loaded onto the front end portion 14, and captures inside of the body cavity through the image capturing window 41 and displays the captured image on the monitor 18. Image displayed on the monitor 18 is recorded to a RAM 44 (see FIG. 4) incorporated in the processing device 20.

The self-propelling device 12 attached to the front end portion 14 carries the front end portion 14, i.e. the insert section 13a forward or backward in the digestive tract. The self-propelling device 12 and the drive source device 16 are connected by a torque wire 37 in the protective sheath 36, and rotation torque of the drive source device 16 is transmitted to the self-propelling device 12 via the torque wire 37.

In FIG. 4, the drive source device 16 rotates the motor 45 according to command signal from the controller 17, and transmits rotation of the motor 45 to the self-propelling device 12 via the torque wire 37. Moreover, the drive source device 16 incorporates a speaker 46, and emits sound from the speaker when the self-propelling device 12 is driven, and informs that the self-propelling device 12 is driven, i.e. the front end portion 14 of the endoscope 15 is moving. Different sounds are used for informing respectively forward movement and backward movements of the self-propelling device 12. Moreover, the tone or volume of the sound may be changed according to moving velocity of the self-propelling device 12. The speaker 46 can be turned off by a switch (not shown).

Next, the operation of the endoscopy system 10 will be described now. The insert section 13a is inserted into the digestive tract such as large intestine, the condition of inner wall of the digestive tract is captured through the image capturing window 41 provided at the front end portion 14, and the captured image is displayed on a display screen 50 of the monitor 18.

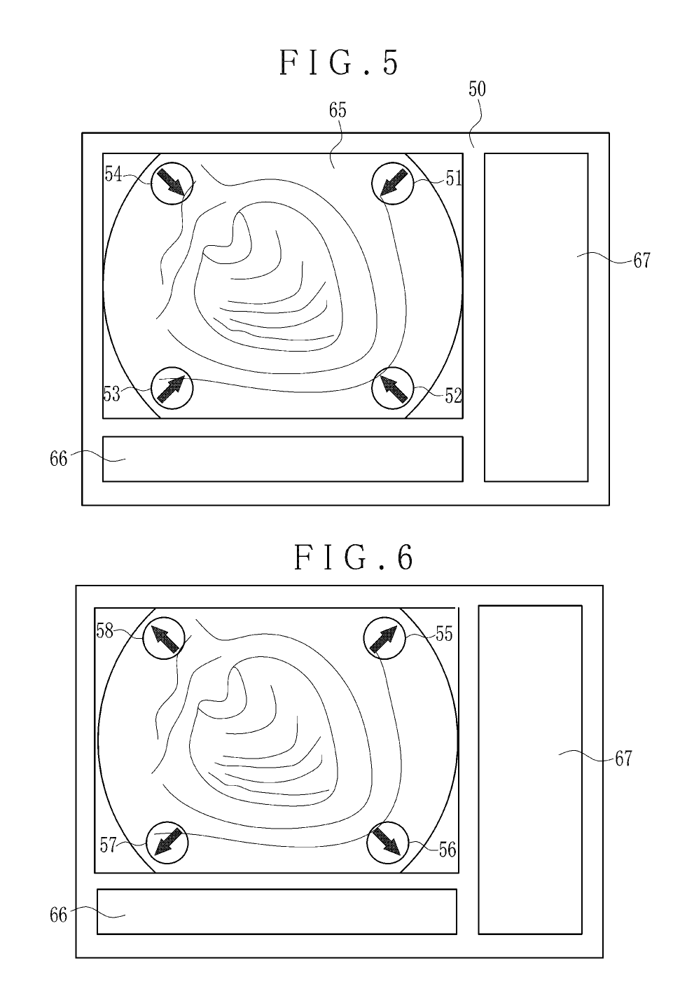

In FIG. 5, when the forward button 24 of the controller 17 is operated, the front end portion 14 is carried forward in the digestive tract by the self-propelling device 12, and drive information of the drive source device 16 is displayed on the monitor 18 as the moving information of the front end portion 14. This drive information is displayed with arrows 51 to 54 at each four corner in an image display area 65 provided in the display screen 50. The arrows 51 to 54 are displayed inward so that each arrow located at four corners of the image display area 65 points center respectively. At this time, when the speaker 46 is turned on, the sound for showing forward movement is informed from the speaker 46.

In FIG. 6, when the backward button 25 of the controller 17 is operated, the front end portion 14 moves backward in the digestive tract by the self-propelling device 12. As the moving information of the front end portion 14, information from the drive source device 16 is displayed by arrows 55 to 58 at four corners in the image display area 65 of the monitor 18. The arrows 55 to 58 are displayed so that each arrow located at four corners of the image display area 65 respectively points outward from the center. At this time, when the speaker 46 is turned on, sound showing backward movement is informed from the speaker 46.

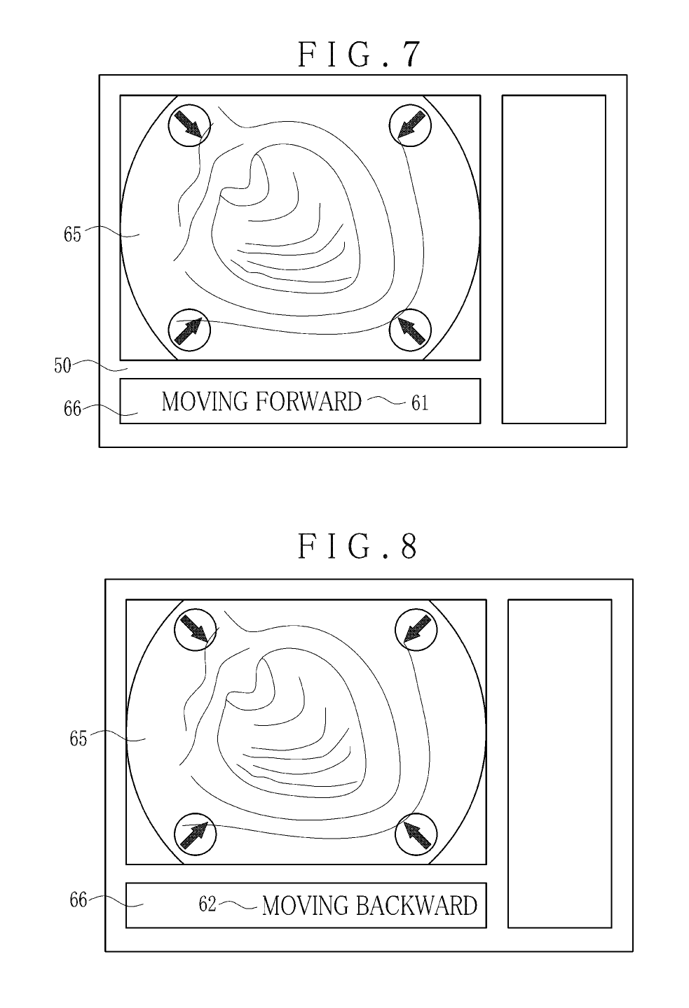

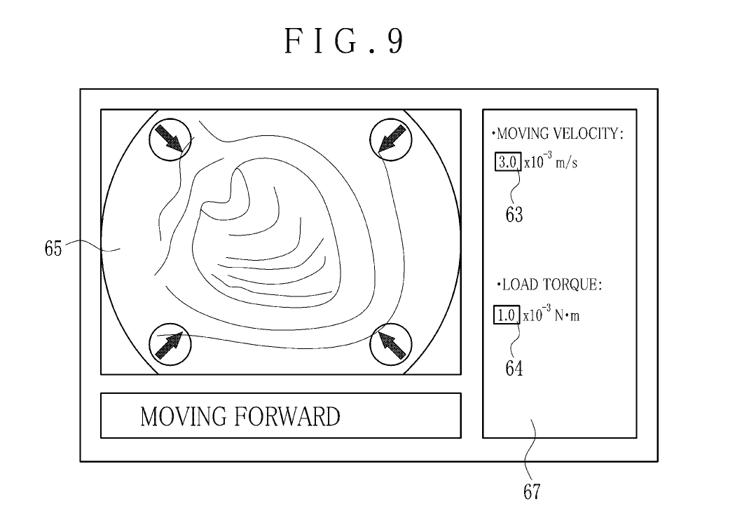

As shown in FIG. 7 or FIG. 8, the moving forward 61 or the moving backward 62 may be displayed in the textual display area 66 provided in the display screen 50 of the monitor 18, as moving information of the front end portion 14. Further, as shown in FIG. 9, it may be possible that moving velocity of the self-propelling device 12 calculated from the rotation of the motor 45 and the load torque loaded to the self-propelling device 12 are transferred to the processing device 20 from the drive source device 16 to display moving velocity 63 and load torque 64 on a textual display area 67.

The arrows 51 to 58 displayed on the image display area 65, moving forward 61, moving backward 62, the moving velocity 63 and load torque 64 displayed on the textual display areas 66 and 67 are recorded in the RAM 44 incorporated in the processing device 20 as image information with the observed image.

In the aforementioned embodiments, the speaker 46 may be located at the position except for where the drive source device 16 is located, and record of image displayed on the monitor 18 including drive information may be recorded in a recording device except for the RAM 44 incorporated in the processing device 20.

In the aforementioned embodiments, the self-propelling device having toroid described in WO2007/050370 is used, but other self-propelling devices, such as a self-propelling device using an endless belt may be used. Further, in addition to detachable attachment of the self-propelling device 12 to the front end portion 14, the self-propelling device 12 may be built in the front end portion 14.

Various changes and modifications are possible in the present invention and may be understood to be within the present invention.

What is claimed is:

1. An endoscopy system comprising:
a self-propelling endoscope which is self-propulsive in a body cavity, including an insert section inserted into the body cavity, a self-propelling device provided at a front end portion of said insert section, an image capturing section contained in said front end portion, said self-propelling device providing self-propulsion to said insert section to perform forward movement or backward movement, and said image capturing section capturing image of inside of said body cavity;
a drive source device for supplying drive force for self-propulsion to said self-propelling device;
a controller for controlling operation of said self-propelling device through a drive source device; and
a monitor for displaying an observed image captured at said image capturing section and drive information of said drive source device.

2. An endoscopy system according to claim 1, wherein said drive information is displayed on said monitor with said observed image.

3. An endoscopy system according to claim 2, further comprising a recording device for recording a screen displayed on said monitor.

4. An endoscopy system according to claim 2, wherein said drive information includes said forward movement and said backward movement, and they are displayed by at least one of arrow and character.

5. An endoscopy system according to claim 4, wherein said arrows are displayed at four corners of an image display area on which said observed image is displayed.

6. An endoscopy system according to claim 5, wherein four arrows displayed at four corners of said image display area direct toward a center part of said image display area when drive force toward said front end portion is in the direction of forward movement, and said four arrows direct toward outside of said image display area when drive force toward said front end portion is in the direction of backward movement.

7. An endoscopy system according to claim 2, wherein drive information includes at least one of moving velocity and load torque of said front end portion corresponding to said drive force.

8. An endoscopy system according to claim 7, further comprising an alarm for informing said forward movement and said backward movement by different sounds.
